(19) United States
(12) Patent Application Publication
(10) Pub. No.: US 2015/0088267 A1
(43) Pub. Date: Mar. 26, 2015

(54) BONE AUGMENT INTERLOCKING WITH BONE PLATE

(71) Applicant: Frank A. LIPORACE, Englewood Cliffs, NJ (US)

(72) Inventors: Frank A. Liporace, Englewood Cliffs, NJ (US); George J. Haidukewych, Orlando, FL (US); Richard S. Yoon, New York, NY (US)

(21) Appl. No.: 14/385,974

(22) PCT Filed: Mar. 15, 2013
(86) PCT No.: PCT/IB2013/000818

§ 371 Date: Sep. 17, 2014

Related U.S. Application Data
(60) Provisional application No. 61/612,701, filed on Mar. 19, 2012.

(51) Int. Cl. A61F 2/28

(52) U.S. Cl. CPC A61F 2/28; A61F 2002/2835
USPC 623/23.5

(57) ABSTRACT

A bone augment for filling a void in a bone and providing secure fixation of an implant to the bone includes a sidewall structure having a top surface, a first end and a second end. The sidewall structure rises from a base of the bone augment. The first end of the bone augment is disposed opposite to the second end of the bone augment. An insertion aperture is disposed within the bone augment and extends from the first end of the bone augment to the second end of the bone augment. The bone augment receives a bone plate within the insertion aperture and the bone plate fixes the bone augment within the void.

### PRIORITY

This application claims priority to U.S. Provisional Application No. 61/612,701, filed with the United States Patent and Trademark Office on Mar. 19, 2012, the entire contents of which are incorporated herein by reference.

### BACKGROUND OF THE INVENTION

1. Field of the Invention

The present invention relates generally to surgical devices, and more particularly, to a bone augment for interlocking with a bone plate.

2. Brief Description of the Related Art

In the United States, the number of total joint replacements is increasing every year. In 2010, an estimated 500,000 patients received total hip or knee replacements and this does not include those receiving total shoulder, elbow or ankle replacements. The number of total joint replacements will continue to increase as the elderly population grows. Additionally, as engineering improves longevity of prosthetic implants, indications for patients will broaden, driving the age of implantation to younger populations.

A bone void can occur in a variety of settings, including fractures requiring total joint replacement as well as high-energy trauma. Prosthetic implants are generally used to treat such fractures. Bone voids occur in areas surrounding prosthetic implants, often resulting from metal or plastic wear, causing an osteolytic reaction leading to bone resorption and loss. The rise in patients requiring prosthetic implantations will result in an increase in infections, mechanical failures and periprosthetic fractures that lead to creation of bone voids. Inserting a prosthetic implant across a bone void can result in an unstable setting. Currently, the options for fixating fractures across bone voids are limited.

FIG. 1 illustrates an implant 100 extending from a femur 110 that is to be inserted into a cup area 115 of a hip bone 120, in the case of a typical hip replacement. However, the hip bone 120 demonstrates an incidence of bone loss, i.e. bone void 130, such that when the implant 100 is inserted into the cup area 115, stable fixation will not occur because the implant 100 is not secured to the entire surface of the cup area 115.

### SUMMARY OF THE INVENTION

According to an embodiment of the present invention a bone augment is provided for filling a void in a bone and providing secure fixation of an implant to the bone. The bone augment includes a sidewall structure having a top surface, a first end and a second end. The sidewall structure rises from a base of the bone augment. The first end of the bone augment is disposed opposite to the second end of the bone augment. An insertion aperture is disposed within the bone augment and extends from the first end of the bone augment to the second end of the bone augment. The bone augment receives a bone plate within the insertion aperture and the bone plate fixes the bone augment within the void.

### BRIEF DESCRIPTION OF THE DRAWINGS

The above and other objects, features and advantages of certain embodiments of the present invention will be more apparent from the following detailed description taken in conjunction with the accompanying drawings, in which:

FIG. 1 illustrates a diagram of an implant extending from a femur being inserted into a cup area of a hip bone, in a typical hip replacement;

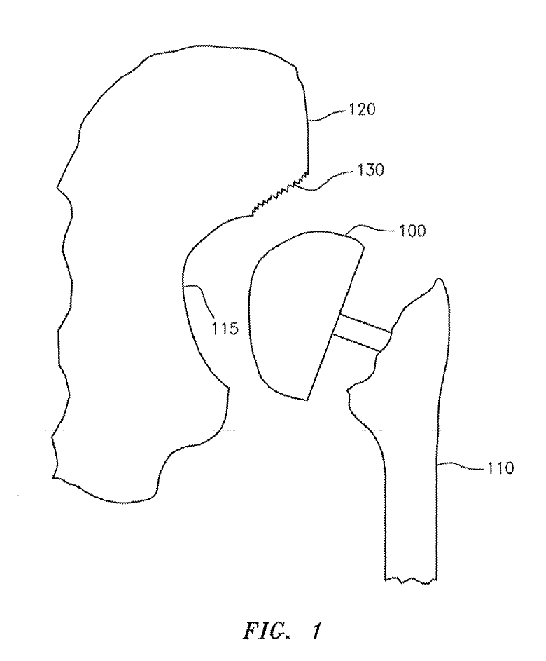

FIG. 2 illustrates a perspective view of a bone augment for filling a void in a bone and providing secure fixation of an implant to the bone, according to an embodiment of the present invention;

FIG. 3 illustrates a cross-sectional view of a bone plate inserted within a bone augment and affixed to a bone, according to an embodiment of the present invention;

FIG. 4 illustrates a top view of a bone plate inserted within a bone augment, according to an embodiment of the present invention;

FIG. 5 illustrates a perspective view of a bone augment including screw-receiving protrusions, according to an embodiment of the present invention;

FIG. 6 illustrates a front view of a bone augment including a bone augment cap, according to an embodiment of the present invention; and

FIG. 7 illustrates a sagittal view of an ilium, acetabulum, pubis, and ischium including a bone plate and a bone augment for treatment of a bone void, according to an embodiment of the present invention.

### DETAILED DESCRIPTION OF EMBODIMENTS OF THE PRESENT INVENTION

The following detailed description of embodiments of the invention will be made in reference to the accompanying drawings. In describing the invention, explanation about related functions or constructions known in the art are omitted for the sake of clearness in understanding the concept of the invention to avoid obscuring the invention with unnecessary detail.

A bone augment is provided for bone fixation in setting a fracture in native bone, periprosthetic bone loss, or where there is a risk of future bone loss. The bone augment may be utilized to fill a void in any bone. The bone augment provides treatment for filling a void in a bone with concurrent skeletal fixation by interlocking with a bone plate.

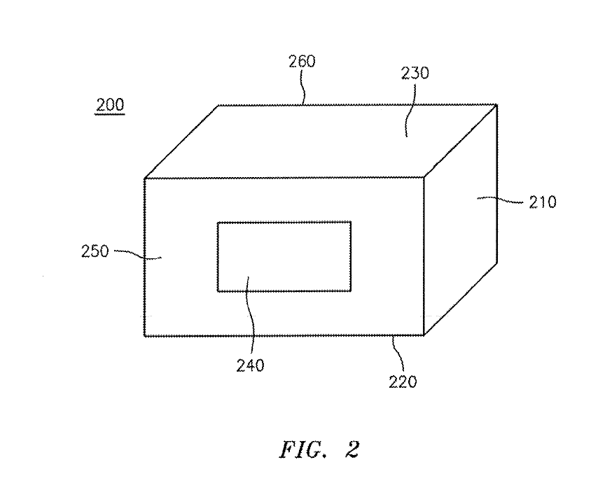

FIG. 2 illustrates a perspective view of a bone augment for filling a void in a bone and providing secure fixation of an implant to the bone, according to an embodiment of the present invention. Bone augment 200 includes a sidewall structure 210. The sidewall structure 210 includes a top surface 230, a first end 250 and a second end 260. Thus, the top surface 230, the first end 250 and the second 260 indicate relative locations of the sidewall structure 210. The sidewall structure 210 rises from a base 220 of the bone augment 200. According to an embodiment of the present invention, the sidewall structure 210 is configured as a rounded surface arising from the base 220, the base 220 having a flat surface. For example, the shape of the bone augment 200 may be embodied as a half-sphere.

The first end 250 of the bone augment 200 is disposed opposite to the second end 260 of the bone augment 200. An insertion aperture 240 is disposed within the bone augment 200 and extends from the first end 250 to the second end 260. The insertion aperture 240 provides an opening in the bone augment 200 that receives a bone plate and allows the bone plate to pass through the bone augment 200. According to an embodiment of the present invention, the insertion aperture 240 extends through the center of the bone augment 200, i.e., the insertion aperture 240 remains equidistant to the sidewall structure 210, the base 220, and the top surface 230 as the insertion aperture 240 extends through the bone augment 200. The bone plate fixes the bone augment 200 within a void in a bone, allowing stable fixation of an implant to the bone.

According to an embodiment of the present invention, the insertion aperture 240 may extend through the bone augment 200 in a concave or a convex manner with respect to the top surface 230. Thus, the insertion aperture 240 is centered in the bone augment 200 at the first end 250 and the second 260, but within the bone augment 200, the insertion aperture 240 may extend in a curved manner closer to the base 220 when concave and closer to the top surface 230 when convex. Similarly, the insertion aperture 240 can be concave or convex with respect to the sidewall structure 210.

Additionally, where the insertion aperture 240 is concave with respect to the top surface 230, openings of the insertion aperture 240 may extend through the top surface 230. In this configuration, the openings of the insertion aperture 240 receive the bone plate perpendicular to a surface of the top surface 230.

Where the insertion aperture 240 is convex with respect to the top surface 220, openings of the insertion aperture 240 may extend through the base 220. In this configuration, the openings of the insertion aperture 240 receive the bone plate perpendicular to the surface of the base 220.

The bone augment 200 is constructed of a porous substrate that allows ingrowth of the bone. The porous substrate may be a porous metal specifically designed for medical implantation. For example, the porous substrate may be tantalum, or a tantalum alloy. Use of the tantalum alloy allows for the size and shape of the augment 200 to be adjusted prior to implantation, i.e., the sidewall structure 210 and the base 220 of the augment 200 can be molded to fit the bone void during the surgical procedure. According to an embodiment of the present invention, the top surface 230 is contoured to receive the particular implant in use.

The porosity of the porous substrate allows bony ingrowth into the augment 200, providing increased adherence of the bone to the bone augment 200 and, thus, improved stability for filling the void in the bone. According to an embodiment of the present invention, the augment 200 includes a plurality of pre-formed pores. The pores may be formed in the sidewall structure 210 and the base 220.

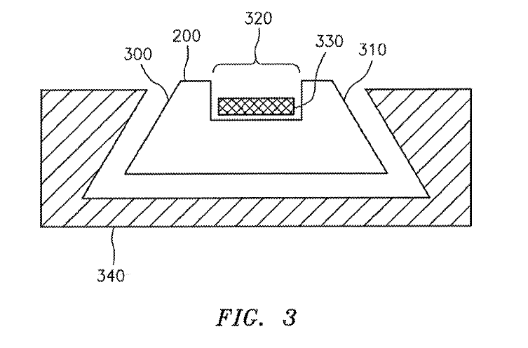

FIG. 3 illustrates a cross-sectional view of a bone plate inserted within a bone augment and affixed to a bone, according to an embodiment of the present invention. The sidewall structure 210 includes a first sidewall 300 and a second sidewall 310 extending from the base 220 to meet the top surface 230. An open channel 320 extends through the top surface 230 from the first end 250 to the second end 260 of the bone augment 200. The channel 320 provides access to the insertion aperture 240 through the top surface 230 of the bone augment 200. Thus, the bone plate 330 may be inserted within the insertion aperture 240 through the first end 250 and out of the second end 260 of the bone augment 200. Alternatively, the bone plate 330 may be inserted within the insertion aperture 240 through the channel 320. The channel 320 may be wide enough to allow insertion of screws through the bone plate 330, through the bone augment 200, and into a bone 340 at varying angles.

The bone augment 200 may include pre-drilled screw holes (not shown). The pre-drilled screw holes may be threaded to provide additional locking. The pre-drilled screw holes may be provided with up to 15 degrees of freedom. Thus, the screws may be inserted into the pre-drilled screw holes in any direction, providing a variety of predetermined fixation angles. However, the bone augment 200 does not require pre-drilled screw holes. Specifically, using a multi-point contact threaded screw head and a differential stiffness metal for the screw and the bone plate, such as titanium, or a cobalt-chromium screw with a titanium bone plate, the screws can cut a screw hole tract in the bone augment 200 allowing for variable angle locking that is not predetermined.

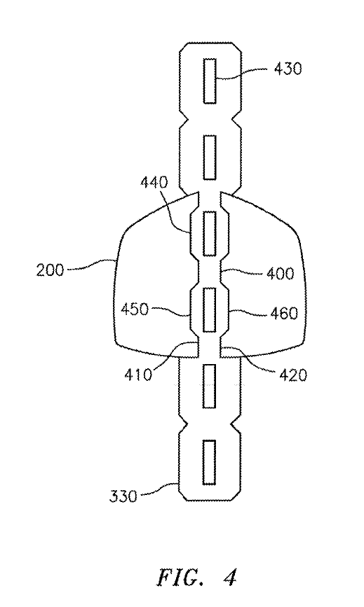

FIG. 4 illustrates a top view of a bone plate inserted within a bone augment, according to an embodiment of the present invention. Specifically, the augment 200 includes a fastener prong 400 extending from the top surface 230 across the insertion aperture 240. A length of the fastener prong 400 may be less than a width of the channel 320, thus leaving the channel 320 open to the insertion aperture 240. However, according to an embodiment of the present invention, the fastener prong 400 may extend entirely across the insertion aperture 240, partially enclosing a portion of the top surface 230 of the bone augment 200 around the bone plate 330.

The fastener prong 400 includes a first fastener prong 410 disposed opposite to a second fastener prong 420 on the top surface 230. The first fastener prong 410 and the second fastener prong 420 extend towards a middle portion of the insertion aperture 240 to a point above and/or below screw insertion openings 430 of the bone plate 330 when the bone plate 330 is inserted within the insertion aperture 240. The first fastener prong 410 and the second fastener prong 420 secure the bone plate 330 within the insertion aperture 240. According to an embodiment of the present invention, the bone augment 200 includes additional fastener prongs, i.e., third, fourth, fifth and sixth fastener prongs etc., that secure the bone plate 330 within the insertion aperture 240 along the entire length of the bone augment 200.

The augment 200 includes a plurality of recessed portions 440 in the top surface 230. The plurality of recessed portions 440 extend across the channel 320 a distance less than a length of the fastener prong 400. The recessed portions 440 include a first recessed portion 450 and a second recessed portion 460 extending across the insertion aperture 240. According to an embodiment of the present invention, the first recessed portion 450 and the second recessed portion 460 extend the top surface 230 around the bone plate 330 across the insertion aperture 240 to a point less than the fastener prong 400.

FIG. 4 further illustrates that the bone plate 330 is aligned within the insertion aperture 240 such that the first recessed portion 450 and the second recessed portion 460 allow access to the openings 430 of the bone plate 330. That is, the first recessed portion 450 and the second recessed portion 460 extend across the insertion aperture 240 to a point where variable-angled insertion of bone screws through the openings 430 remains possible. The first recessed portion 450 and the second recessed portion 460 may extend farther across the insertion aperture 240 when the first recessed portion 450 and the second recessed portion 460 gradually reduce in thickness while extending across the insertion aperture 240. Thus, according to an embodiment of the present invention, a thickness of the plurality of recessed portions 440 and a thickness of the fastener prong 400 decreases as the plurality of recessed portions 440 and the fastener prong 400 extends across the insertion aperture 240. Decreasing thickness of the plurality of recessed portions 440 and the fastener prong 400 allows variable angled insertion of bone screws through the bone plate 330 and into the bone augment 200. The thickness of the plurality of recessed portions 440 and the fastener prong 400 may be thinnest at the middle portion of the insertion aperture 240. The first fastener prong 410, the second fastener prong 420, the first recessed portion 450 and the second recessed portion 460 may similarly gradually reduce in thickness while extending across the insertion aperture 240.

The openings 430 of the bone plate 330 allow insertion of screws, i.e., locking or non-locking elements/fasteners. The screws may be a statically locked element or a variable-angled locked element for securing the bone plate 330 and the bone augment 200 to the bone 340. The bone plate 330 and the bone augment 200 may be coated by a resorbable or non-resorbable substrate for delivering medication including antibiotics and growth factors. Thus, the bone augment 200 providing a vehicle for local delivery of medication.

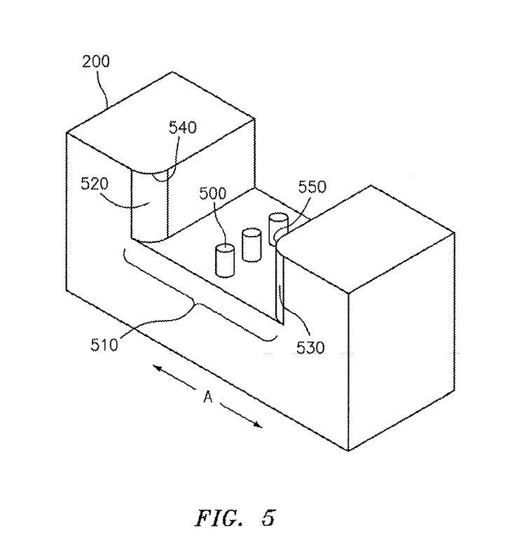

FIG. 5 illustrates a perspective view of a bone augment including screw-receiving protrusions, according to an embodiment of the present invention. The bone augment 200 includes screw-receiving protrusions 500 that extend from within the insertion aperture 240. The screw-receiving protrusions 500 rise from a bottom surface of the insertion aperture 240 in a perpendicular manner. The screw-receiving protrusions 500 extend into the openings 430 of the bone plate 330 when the bone plate 330 is inserted into the insertion aperture 240. Thus, the bone plate 330 is locked into the bone augment 200, preventing movement of the bone plate 330 while the bone plate 330 is screwed into the bone 340.

FIG. 5 further illustrates the insertion aperture 240 having an expanded opening 510 at the first end 250 of the bone augment 200. The expanded opening 510 includes a first arcuate surface 520 and a second arcuate surface 530. The first arcuate surface 520 and the second arcuate surface 530 extend from the insertion aperture 240 to meet the first end 250 of the bone augment 200. The first arcuate surface 520 includes a first arcuate rim 540 extending along an upper edge of the top surface 230 from the insertion aperture 240 to the first end 250. The second arcuate surface 530 includes a second arcuate rim 550 extending along the upper edge of the top surface 230 from the insertion aperture 240 to the first end 250. A width of the expanded opening 510 is greater than a width of the insertion aperture 240. Accordingly, when a portion of the bone plate 330 is inserted within the insertion aperture 240, portions of the bone plate 330 outside of the bone augment 200 can be moved laterally with respect to the bone augment 200 in the direction of arrow A. Positioning of the bone plate 330 can therefore be adjusted, allowing proper fixation of the bone plate 330 to the bone 340.

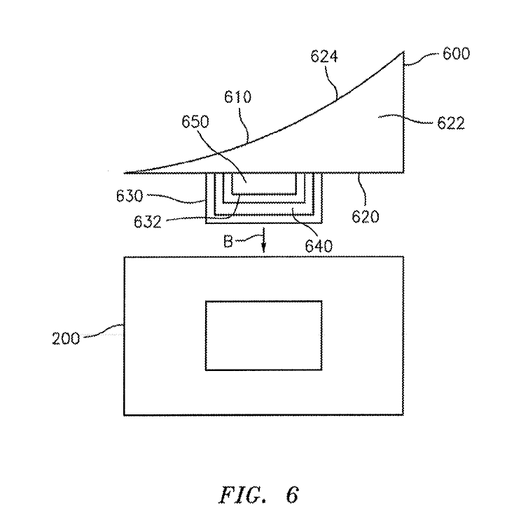

FIG. 6 illustrates a front view of a bone augment including a bone augment cap, according to an embodiment of the present invention. A bone augment cap 600 includes an implant receiving surface 610 and a cap base 620. The bone augment cap 600 may be constructed of a porous substrate, similar to the bone augment 200. However, since the bone augment cap 600 is not in contact with the bone 340, the bone augment cap 600 may be constructed of any material suitable for medical implantation. The implant receiving surface 610 rises from the cap base 620. The implant receiving surface 610 is provided in a shape that contours to receive a particular implant, thus providing a stable environment for fixation of the implant to the bone 340. The contour of the implant receiving surface 610 illustrated in FIG. 6 is provided for illustrative purposes only. That is, the implant receiving surface's 610 contour may vary depending on the particular implant in use.

The bone augment cap 600 further includes a first cap end 622 and a second cap end 624. The first cap end 622 is disposed opposite to the second cap end 624. A first cap flange 630 extends from the cap base 620 on the first cap end 622. A second cap flange 632 extends from the cap base 620 on the second cap end 624. The first cap flange 630 includes a first bone plate-receiving aperture 640 and the second cap flange 632 includes a second bone plate-receiving aperture 650. When the bone augment cap 600 is attached to the bone augment 200 in the direction of arrow B, the cap base 620 rests on the top surface 230 and the first and second bone plate-receiving apertures 640 and 650 overlap with the insertion aperture 240. Thus, the bone plate 330 is inserted through the first bone plate-receiving aperture 640, through the bone augment 200 via the insertion aperture 240, and out through the second bone plate-receiving aperture 650, locking the bone augment cap 600 to the bone augment 200. Accordingly, the bone augment cap 600 contacts the top surface 230 and interlocks with the bone augment 200 when the bone plate 330 is inserted within the insertion aperture 240.

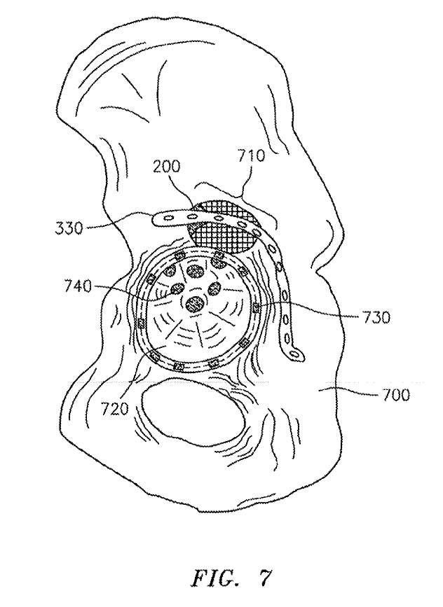

FIG. 7 illustrates a sagittal view of an ilium, acetabulum, pubis, and ischium including a bone plate and a bone augment for treatment of a bone void, according to an embodiment of the present invention. The bone plate 330 is fixed to a hip bone 700. The bone augment 200 fills a bone void 710 in the hip bone 700. The bone plate 330 attaches the bone augment 200 to the hip bone 700 with bone screws inserted through the bone plate openings 430.

FIG. 7 further illustrates an acetabular component 720, i.e., a socket portion of a hip replacement, of a total hip arthroplasty revision where a failed total hip arthroplasty resulted in the bone void 710 requiring use of the bone augment 200. The bone void 710 can also occur as a result of trauma or chronic disease. The acetabular component 720 includes locking tines 730, which lock-in a polyethylene liner (not shown) over the acetabular component 720. The acetabular component 720 also includes component screw holes 740 for fixation of locking screws flush with a surface of the hip bone 700.

In the embodiment illustrated in FIG. 7, the bone augment 200 is inserted above and behind the acetabular component 720. Thus, the bone plate 330 locks the bone augment 200 into the bone void 710, providing a stable base for the acetabular component 720 to be fixed to the hip bone 700.

While the invention has been shown and described with reference to certain embodiments of the present invention thereof, it will be understood by those skilled in the art that various changes in details may be made therein without departing from the spirit and scope of the present invention as defined by the appended claims and equivalents thereof.

What is claimed is:

1. A bone augment for filling a void in a bone and providing secure fixation of an implant to the bone, comprises:
a sidewall structure comprising a top surface, a first end and a second end of the bone augment, the sidewall structure arising from a base of the bone augment, wherein the first end of the bone augment is disposed opposite to the second end of the bone augment; and
an insertion aperture disposed within the bone augment and extending from the first end of the bone augment to the second end of the bone augment,
wherein the bone augment receives a bone plate within the insertion aperture and the bone plate fixes the bone augment within the void.

2. The bone augment of claim 1, a channel extending through the top surface from the first end of the bone augment to the second end of the bone augment.

3. The bone augment of claim 2, wherein the channel provides access to the insertion aperture through the top surface of the bone augment.

4. The bone augment of claim 1, further comprising a fastener prong extending from the top surface across the insertion aperture.

5. The bone augment of claim 4, wherein a length of the fastener prong is less than a width of the channel.

6. The bone augment of claim 4, wherein the fastener prong extends across the insertion aperture, enclosing a portion of the top surface of the bone augment around the bone plate.

7. The bone augment of claim 4, further comprising a plurality of recessed portions in the top surface of the bone augment.

8. The bone augment of claim 7, wherein the plurality of recessed portions extend across the channel a distance less than a length of the fastener prong.

9. The bone augment of claim 8, wherein a thickness of the plurality of recessed portions and a thickness of the fastener prong decreases as the plurality of recessed portions and the fastener prong extends across the insertion aperture, allowing variable angled insertion of bone screws.

10. The bone augment of claim 1, further comprising a plurality of pores.

11. The bone augment of claim 1, further comprising screw-receiving protrusions rising from within the insertion aperture.

12. The bone augment of claim 1, further comprising an expanded opening of the insertion aperture such that a width of the expanded opening is greater than a width of the insertion aperture.

13. The bone augment of claim 12, wherein the expanded opening comprises a first arcuate surface extending from the insertion aperture to the first end and a second arcuate surface extending from the insertion aperture to the first end.

14. The bone augment of claim 1, further comprising:
a bone augment cap contacting the top surface and interlocking with the bone augment when the bone plate is inserted within the insertion aperture.

15. The bone augment of claim 13, wherein the bone augment cap further comprises:
an implant receiving surface arising from a cap base;
a first cap end disposed opposite to a second cap end;
a first cap flange extending from the cap base on the first cap end; and
a second cap flange extending from the cap base on the second cap.

16. The bone augment of claim 14, wherein the first cap flange comprises a first bone plate-receiving aperture and the second cap flange comprises a second bone plate-receiving aperture.

17. The bone augment of claim 15, wherein the cap base rests on the top surface and the first and second bone plate-receiving apertures overlap with the insertion aperture when the bone augment cap is attached to the bone augment and the bone plate is inserted through the first bone plate-receiving aperture, through the bone augment via the insertion aperture and out through the second bone plate-receiving aperture, locking the bone augment cap to the bone augment.

18. The bone augment of claim 1, wherein the bone augment is constructed of a porous substrate.
